(19) United States
(12) Patent Application Publication
(10) Pub. No.: US 2004/0110788 A1
(43) Pub. Date: Jun. 10, 2004

(54) Nicotinic acetylcholine receptor ligands

(76) Inventors: Kai Schiemann, Darmstadt (DE); Joachim Leibrock, Pfungstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/474,801

(22) PCT Filed: Mar. 13, 2002
(86) PCT No.: PCT/EP02/02729

(30) Foreign Application Priority Data
Apr. 14, 2001 (DE) 10118551.0

(51) Int. Cl. C07D 451/02; A61K 31/46

(52) U.S. Cl. 514/304; 546/124; 546/125

(57) ABSTRACT

Compounds of the formula I

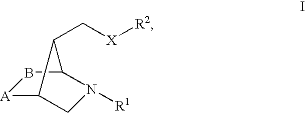

in which A-B, X, R¹ and R² are as defined in claim 1, are ligands of the nicotinic acetylcholine receptor and are suitable for the prophylaxis or treatment of schizophrenia, depression, anxiety states, dementia, Alzheimer's disease, Lewy bodies dementia, neurodegenerative disorders, Parkinson's disease, Huntington's disease, Tourette's syndrome, learning and memory restrictions, age-induced memory impairment, amelioration of withdrawal symptoms in nicotine dependence, strokes or brain damage by toxic compounds.

The invention relates to (2-azabicyclo[2.2.1]hept-7-yl)methyl and (2-azabicyclo[2.2.1]hept-5-en-7-yl)methyl derivatives of the formula I

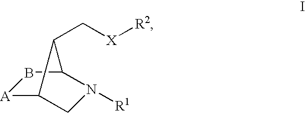

in which

A-B is a single or double bond,

X is O, NR³ or S,

R¹ is hydrogen, A, Ar, arylalkyl, Het, C(O)—R⁴, SO₂—R⁴, C(S)N(R⁴)₂ or COOR⁴,

R² is A, Ar, arylalkyl, Het, C(O)—R⁴, SO₂—R⁵, C(S)N(R⁵)₂ or COOR⁴,

R³ to R⁵ are each, independently of one another, hydrogen, A, cycloalkyl, Ar or arylalkyl,

R⁶ is hydrogen or A,

A is a linear or branched alkyl group having from 1 to 10 carbon atoms,

Ar is phenyl, naphthyl or biphenyl, each of which is unsubstituted or monosubstituted or polysubstituted by Hal, A, OR⁶, N(R⁶)₂, NO₂, CN, COOR⁵, CON(R⁶)₂, NR⁶COR⁶, NR⁶CON(R⁸)₂, NR⁶SO₂A, COR⁶, SO₂NR⁶, S(O)ₘA or Het¹,

arylalkyl is arylalkyl having 7-14 carbon atoms,

cycloalkyl is cycloalkyl having from 3 to 10 carbon atoms,

Hal is F, Cl, Br or I,

Het is a saturated, unsaturated or aromatic monocyclic or bicyclic heterocyclic radical having from 5 to 10 ring members which may contain from 1 to 4 N and/or from 1 to 4 S and/or from 1 to 4 O atoms, and where the heterocyclic radical may be monosubstituted, disubstituted or trisubstituted by Hal, A, —[C(R⁶)₂]ₒ—Ar, —[C(R⁶)₂]ₒ-cycloalkyl, OR⁶, N(R⁶)₂, NO₂, CN, COOR⁶, CON(R⁶)₂, NR⁶COA, NR⁶CON(R⁶)₂, NR⁶SO₂A, COR⁶, SO₂NR⁶ or S(O)ₘA and/or carbonyl oxygen,

Het¹ is 3-methyl-2,5-dioxopyrrolidin-1-yl or 1,3-dioxo-1,3-dihydroisoindol-2-yl,

m is 1 or 2,

o is 0, 1, 2, 3 or 4,

and physiologically acceptable salts and solvates thereof.

Similar compounds are disclosed in WO 92/05172.

The invention had the object of finding novel compounds having valuable properties, in particular those which can be used for the preparation of medicaments.

It has been found that the compounds of the formula I and their physiologically acceptable salts and solvates are well tolerated and have valuable pharmacological properties since they act on the central nervous system. The compounds are nicotinic acetylcholine receptor ligands.

Of the well-characterised class of acetylcholine receptors, some members have been implicated in certain disorders of the central nervous system. Known active ingredients which are able to interact with the acetylcholine receptor class are, for example, pilocarpine, nicotine, lobeline and epibatidine.

These nicotinic acetylcholine receptors can be divided into two main classes, depending on the sites at which they occur.

The first class comprises the neuromuscular receptors. These are sub-divided into (α₁α₁βεδ) and (α₁α₁βγδ) receptors. The second class comprises the neuronal nicotinic acetylcholine receptors, which are found in the ganglia. In these, a distinction is made between the (β₂-β₅) receptors and the (α₂-α₉) receptors, in this respect see also “Basic Neurochemistry”, Ed. Siegel et al., Raven Press, New York, 1993.

The substances of the formula I are capable of interacting with each of these receptors. The substances of the formula I interact particularly well with the nicotinic α₇ receptor.

In-vitro evidence of the interaction with the nicotinic α₇ receptor can be obtained, for example, analogously to J. M. Ward et al., FEB 1990, 270, 45-48 or D. R. E. Macallan, FEB 1998, 226, 357-363.

Further in-vitro tests for nicotinic receptors are described in F. E. D'Amour et al., Manual for Laboratory Work in Mammalian Physiology, 3rd Ed., The University of Chicago Press (1965), W. Sihver et al., Neuroscience 1998, 85, 1121-1133 or B. Latli et al., J. Med. Chem. 1999, 42, 2227-2234.

Illnesses which can be treated with the substances of the formula I include schizophrenia, depression, anxiety states, dementia, in particular Alzheimer's disease and Lewy bodies dementia, neurodegenerative disorders, Parkinson's disease, Huntington's disease, Tourette's syndrome, learning and memory restrictions, age-induced memory impairment, and amelioration of withdrawal symptoms in nicotine dependence. Owing to their neuroprotective action, compounds of the formula I are used in strokes and brain damage by toxic compounds.

Compounds of the formula I and their salts and solvates are also suitable as intermediates for the preparation of other medicament active ingredients.

The invention relates to the compounds of the formula I and to their physiologically acceptable acid-addition salts. The invention also relates to the solvates, for example hydrates or alcoholates, of these compounds.

The term “solvates of the compounds of the formula I” is taken to mean adducts of inert solvent molecules onto the compounds of the formula I which form owing to their mutual attractive force. Solvates are, for example, monohydrates or dihydrates or addition compounds with alcohols, such as, for example, with methanol or ethanol.

The compounds of the formula I have at least one asymmetrical carbon atom, which can have different configurations. They can therefore exist in various optically active forms or alternatively as racemates. In the case of the compounds of the formula I, the bridgehead carbon atom means that stereoisomers exist, which are known as endo or exo isomers. Exo isomers are compounds of the formula I in which the substituent, i.e. —CH₂—X—R², is further away from the functional group N—R¹.

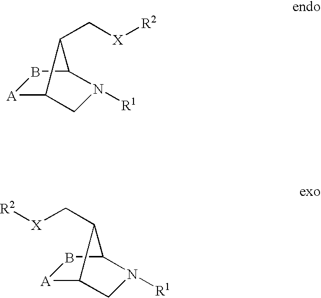

The two stereoisomers are generally in equilibrium at room temperature and therefore cannot be isolated individually.

The generic formulae I and II cover both the exo and endo isomers.

The invention relates to the compounds of the formula I and to salts and solvates thereof according to claim 1 and to a process for the preparation of compounds of the formula I and salts and solvates thereof, characterised in that

a) a compound of the formula II

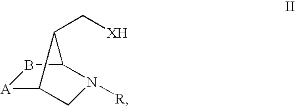

in which A-B is a single or double bond,

X is O, and

R is A, Ar, arylalkyl, Het, C(O)—R⁴, SO₂—R⁴, C(S)N(R⁴)₂, COOR⁴ or an amino-protecting group,

is reacted with a compound of the formula III

L-C(O)—R⁴ III,

in which

R⁴ is as defined in claim 1, with free amino or hydroxyl groups being in protected form during the reaction, and the protecting groups being cleaved off after esterification, and

L is Cl, Br, I or a free or reactively functionally modified OH group, and the radical R is, if desired, converted into a radical R¹, where R¹ is as defined in claim 1, or

b) a compound of the formula II

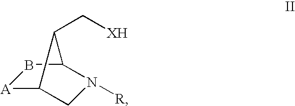

in which A-B is a single or double bond,

X is NR³,

R³ is as defined in claim 1, and

R is A, Ar, arylalkyl, Het, C(O)—R⁴, SO₂—R⁴, C(S)N(R⁴)₂, COOR⁴ or an amino-protecting group,

is reacted with a compound of the formula IV

L-SO₂—R⁵ IV,

in which R⁵ is as defined in claim 1, and

L is Cl, Br, I or a free or reactively functionally modified OH group, and the radical R is, if desired, converted into a radical R¹, where R¹ is as defined in claim 1, or

c) a compound of the formula II

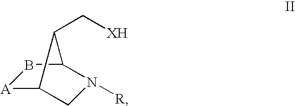

in which A-B is a single or double bond,

X is O or NR³,

R³ is as defined in claim 1, and

R is A, Ar, arylalkyl, Het, C(O)—R⁴, SO₂—R⁴, C(S)N(R⁴)₂, COOR⁴ or an amino-protecting group,

is reacted with a compound of the formula V

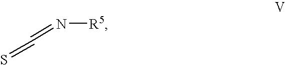

in which R⁵ is as defined in claim 1,

and the radical R is, if desired, converted into a radical R¹, where R¹ is as defined in claim 1, or

d) if desired, one of the radicals R, R¹ and/or a substituent of the aryl group is converted into another radical R, R¹ and/or a substituent of the aryl group by, for example, cleaving an OA group to form an OH group and/or converting a CHO group into a CN group and/or hydrogenating a benzyl group, and/or

a base of the formula I obtained is converted into one of its salts by treatment with an acid.

The invention also relates to the compounds of the formula I according to claim 1 and their physiologically acceptable salts and solvates as medicament active ingredients.

The invention likewise relates to the compounds of the formula I according to claim 1 and their physiologically acceptable salts or solvates as ligands of the nicotinic acetylcholine receptor.

For all radicals which may occur more than once, such as, for example, R⁵, A or Hal, their meanings are independent of one another.

A-B is a single or double bond, the single bond being preferred.

X is O, NR³ or S, where R³ has one of the meanings given below. X is particularly preferably O or NH.

A is linear or branched alkyl having from 1 to 10 carbon atoms and preferably has 1, 2, 3, 4, 5, 6, 7, 8, 9 or 10 carbon atoms. Alkyl having from 1 to carbon atoms is preferably methyl, furthermore ethyl, n-propyl, isopropyl, n-butyl, sec-butyl or tert-butyl, furthermore also n-pentyl, 1-, 2- or 3-methylbutyl, n-hexyl, 1-, 2-, 3- or 4-methylpentyl, n-heptyl, n-octyl, n-nonyl or n-decyl.

Ar is phenyl, naphthyl or biphenyl, each of which is unsubstituted or monosubstituted or polysubstituted by Hal, A, OR⁶, N(R⁶)₂, NO₂, CN, COOR⁶, CON(R⁶)₂, NR⁶COR⁶, NR⁶CON(R⁶)₂, NR⁶SO₂, COR⁶, SO₂NR⁶, SO₂NR⁶, S(O)ₘA or Het¹, where A has one of the meanings indicated above, and R⁶, Het¹ and m have one of the meanings indicated below.

Ar is preferably unsubstituted or substituted phenyl, naphthyl or biphenyl, specifically preferably phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-trifluoromethylphenyl, o-, m- or p-aminophenyl, o-, m- or p-hydroxyphenyl, o-, m- or p-nitrophenyl, o-, m- or p-(trifluoromethoxy)-phenyl, o-, m- or p-cyanophenyl, o-, m- or p-methoxyphenyl, o-, m- or p-ethoxyphenyl, o-, m- or p-fluorophenyl, o-, m- or p-bromophenyl, o-, m- or p-chlorophenyl, o-, m- or p-(difluoromethoxy)phenyl, o-, m- or p-(fluoromethoxy)phenyl, furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2-chloro-3-methyl-, 2-chloro-4-methyl-, 2-chloro-5-methyl-, 2-chloro-6-methyl-, 2-methyl-3-chloro-, 2-methyl-4-chloro-, 2-methyl-5-chloro-, 2-methyl-6-chloro-, 3-chloro-4-methyl-, 3-chloro-5-methyl- or 3-methyl-4-chlorophenyl, 2-bromo-3-methyl-, 2-bromo-4-methyl-, 2-bromo-5-methyl-, 2-bromo-6-methyl-, 2-methyl-3-bromo-, 2-methyl-4-bromo-, 2-methyl-5-bromo-, 2-methyl-6-bromo-, 3-bromo-4-methyl-, 3-bromo-5-methyl- or 3-methyl-4-bromophenyl, 2-amino-5-bromophenyl, 2-amino-5-fluorophenyl, 2-amino-5-chlorophenyl, 2-amino-3-methylphenyl, 2-amino-5-nitrophenyl, 2-amino-4,5-dimethoxyphenyl, 3-methyl-2,5-dioxopyrrolidin-1-yl-phenyl, 5-fluoro-2-(3-methyl-2,5-dioxopyrrolidin-1-yl)-phenyl, 5-bromo-2-(3-methyl-2,5-dioxopyrrolidin-1-yl)phenyl, 2-(3-methyl-2,5-dioxopyrrolidin-1-yl)-5-nitrophenyl, 4-chloro-2-(3-methyl-2,5-dioxopyrrolidin-1-yl)-phenyl, 4,5-dimethoxy-2-(3-methyl-2,5-dioxopyrrolidin-1-yl)phenyl, 2-(1,3-dioxo-1,3-dihydroisoindol-2-yl)phenyl, 2-(1,3-dioxo-1,3-dihydroisoindol-2-yl)-3-methylphenyl, 4-chloro-2-(1,3-dioxo-1,3-dihydroisoindol-2-yl)phenyl, 2-(1,3-dioxo-1,3-dihydroisoindol-2-yl)-5-fluorophenyl, 4,5-dimethoxy-2-(1,3-dioxo-1,3-dihydroisoindol-2-yl)-phenyl, 2,4- or 2,5-dinitrophenyl, 2,5- or 3,4-dimethoxyphenyl, 3-nitro-4-chlorophenyl, 2,3,4-, 2,3,5-, 2,3,6-, 2,4,6- or 3,4,5-trichlorophenyl, 2,4,6-tri-tert-butylphenyl, furthermore preferably 2-nitro-4-(trifluoromethyl)phenyl, 3,5-di-(trifluoromethyl)phenyl, 2,5-dimethylphenyl, 2-hydroxy-3,5-dichlorophenyl, 2-fluoro-5- or 4-fluoro-3-(trifluoromethyl)phenyl, 4-chloro-2- or 4-chloro-3-(trifluoromethyl)-, 2-chloro-4- or 2-chloro-5-(trifluoromethyl)phenyl, 4-bromo-2- or 4-bromo-3-(trifluoromethyl)phenyl, p-iodophenyl, 2-nitro-4-methoxyphenyl, 2,5-dimethoxy-4-nitrophenyl, 2-methyl-5-nitrophenyl, 2,4-dimethyl-3-nitrophenyl, 4-fluoro-3-chlorophenyl, 4-fluoro-3,5-dimethylphenyl, 2-fluoro-4-bromophenyl, 2,5-difluoro-4-bromophenyl, 2,4-dichloro-5-methylphenyl, 3-bromo-6-methoxyphenyl, 3-chloro-6-methoxyphenyl, 2-methoxy-5-methylphenyl or 2,4,6-triisopropylphenyl.

Ar is particularly preferably phenyl, o-aminophenyl, p-methoxyphenyl, 2-amino-5-bromophenyl, 2-amino-5-fluorophenyl, 2-amino-5-chlorophenyl, 2-amino-3-methylphenyl, 2-amino-5-nitrophenyl, 2-amino-4,5-dimethoxyphenyl, 3-methyl-2,5-dioxopyrrolidin-1-yl-phenyl, 5-fluoro-2-(3-methyl-2,5-dioxopyrrolidin-1-yl)phenyl, 5-bromo-2-(3-methyl-2,5-dioxopyrrolidin-1-yl)phenyl, 2-(3-methyl-2,5-dioxopyrrolidin-1-yl)-5-nitrophenyl, 4-chloro-2-(3-methyl-2,5-dioxopyrrolidin-1-yl)phenyl, 4,5-dimethoxy-2-(3-methyl-2,5-dioxopyrrolidin-1-yl)phenyl, 2-(1,3-dioxo-1,3-dihydroisoindol-2-yl)phenyl, 2-(1,3-dioxo-1,3-dihydroisoindol-2-yl)-3-methylphenyl, 4-chloro-2-(1,3-dioxo-1,3-dihydroisoindol-2-yl)phenyl, 2-(1,3-dioxo-1,3-dihydroisoindol-2-yl)-5-fluorophenyl or 4,5-dimethoxy-2-(1,3-dioxo-1,3-dihydroisoindol-2-yl)phenyl.

Arylalkyl having from 7 to 14 carbon atoms is preferably benzyl, phenylethyl, phenylpropyl, phenylbutyl, phenylpentyl, phenylhexyl, phenylheptyl, naphthylmethyl, naphthylethyl, naphthylpropyl or naphthylbutyl. arylalkyl is particularly preferably benzyl.

Cycloalkyl having from 3 to 10 carbon atoms is preferably cyclopropyl, cyclobutyl, cyclopentyl, cyclohexyl, cycloheptyl or cyclooctyl. Cycloalkyl is likewise a monocyclic or bicyclic terpene, preferably p-menthane, menthol, pinane, bornane or camphor, including all known stereoisomeric forms, or adamantyl. For camphor, this is either L-camphor or D-camphor.

Hal is fluorine, chlorine, bromine or iodine, particularly preferably fluorine, chlorine or bromine.

Het is a saturated, unsaturated or aromatic monocyclic or bicyclic heterocyclic radical having from 5 to 10 ring members, which may contain from 1 to 4 N and/or from 1 to 4 S and/or from 0.1 to 4O atoms and in which the heterocyclic radical may be monosubstituted, disubstituted or trisubstituted by Hal, A, —[C(R⁶)₂]ₒ—Ar, —[C(R⁶)₂]ₒ-cycloalkyl, OR⁶, N(R⁶)₂, NO₂, CN, COOR⁶, CON(R⁶)₂, NR⁶COA, NR⁶CON(R⁶)₂, NR⁶SO₂A, COR⁶, SO₂NR⁶ or S(O)ₘA and/or carbonyl oxygen, where A, Hal, Ar and cycloalkyl have one of the meanings indicated above, and R⁶, o and m are as defined below.

Het is preferably substituted or unsubstituted 2- or 3-furyl, 2- or 3-thienyl, 1-, 2- or 3-pyrrolyl, 1-, 2-, 4- or 5-imidazolyl, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, 4- or -5-yl, 1,2,4-triazol-1-, -4- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 2-, 3-, 4-, 5- or 6-2H-thiopyranyl, 2-, 3- or 44H-thiopyranyl, 3- or 4-pyridazinyl, pyrazinyl, 2-, 3-, 4-, 5-, 6- or 7-benzofuryl, 2-, 3-, 4-, 5-, 6- or 7-benzothienyl, 1-, 2-, 3-, 4-, 5-, 6- or 7-1H-indolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 4- or 5-benzothiadiazolyl, 2-, 4-, 5-, 6- or 7-benzisothiazolyl, 4-, 5-, 6- or 7-benz-2,1,3-oxadiazolyl, 1-, 2-, 3-, 4-, 5-, 6-, 7- or 8-quinolinyl, 1-, 3-, 4-, 5-, 6-, 7- or 8-isoquinolinyl, 1-, 2-, 3-, 4- or 9-carbazolyl, 1-, 2-, 3-, 4-, 5-, 6-, 7-, 8- or 9-acridinyl, 3-, 4-, 5-, 6-, 7- or 8-cinnolinyl, 2-, 4-, 5-, 6-, 7- or 8-quinazolinyl. The heterocyclic radicals may also be partially or fully hydrogenated. Het may thus also be 2,3-dihydro-2-, -3-, -4- or -5-furyl, 2,5-dihydro-2-, -3-, 4- or -5-furyl, tetrahydro-2- or -3-furyl, 1,3-dioxolan-4-yl, tetrahydro-2- or -3-thienyl, 2,3-dihydro-1-, -2-, -3-, 4- or -5-pyrrolyl, 2,5-dihydro-1-, -2-, -3-, 4- or -5-pyrrolyl, 1-, 2- or 3-pyrrolidinyl, tetrahydro-1-, -2- or -3-pyrrolyl, tetrahydro-1-, -2- or -4-imidazolyl, 2,3-dihydro-1-, -2-, -3-, -4-, -5-, -6- or -7-1H-indolyl, 2,3-dihydro-1-, -2-, -3-, -4- or -5-pyrazolyl, tetrahydro-1-, -3- or 4-pyrazolyl, 1,4-dihydro-1-, -2-, -3- or -4-pyridyl, 1,2,3,4-tetrahydro-1-, -2-, -3-, -4-, -5- or -6-pyridyl, 1,2,3,6-tetrahydro-1-, -2-, -3-, -4-, -5- or -6-pyridyl, 1-, 2-, 3- or 4-piperidinyl, 1-, 2-, 3- or 4-azepanyl, 2-, 3- or 4-morpholinyl, tetrahydro-2-, -3- or 4-pyranyl, 1,4-dioxanyl, 1,3-dioxan-2-, 4- or -5-yl, hexahydro-1-, -3- or -4-pyridazinyl, hexahydro-1-, -2-, 4- or -5-pyrimidinyl, 1-, 2- or 3-piperazinyl, 1,2,3,4-tetrahydro-1-, -2-, -3-, 4-, -5-, -6-, -7- or -8-quinolinyl, 1,2,3,4-tetrahydro-1-, -2-, -3-, 4-, -5-, -6-, -7- or -8-isoquinolinyl.

Het¹ is 3-methyl-2,5-dioxopyrrolidin-1-yl or 1,3-dioxo-1,3-dihydroisoindol-2-yl.

R¹ is hydrogen, A, Ar, arylalkyl, Het, C(O)—R⁴, SO₂—R⁴, C(S)N(R⁴)₂ or COOR⁴, where A, Ar, arylalkyl and Het have one of the meanings given above, and R⁴ has one of the meanings given below.

R¹ is preferably hydrogen, A or arylalkyl. R¹ is particularly preferably aryl-alkyl.

R² is A, Ar, arylalkyl, Het, C(O)—R⁴, SO₂—R⁵, C(S)N(R⁵)₂ or COOR⁴, where A, Ar, arylalkyl and Het have one of the meanings given above, and R⁴ and R⁵ have one of the meanings given below.

R¹ is preferably A, C(O)—R⁴, SO₂—R⁵ or C(S)N(R⁵)₂.

If X is O, R² is particularly preferably C(O)—R⁴, where R⁴ has one of the meanings given below.

If X is NH, R² is particularly preferably C(O)—R⁴ or SO₂—R⁵, where R⁴ and R⁵ have one of the meanings given below.

R³ and R⁵ are each, independently of one another, hydrogen, A, cycloalkyl, Ar or arylalkyl, where A, cycloalkyl, Ar or arylalkyl have one of the meanings given above.

R³ is preferably hydrogen or alkyl having from 1 to 10 carbon atoms, as described above. R³ is particularly preferably hydrogen.

R⁴ is preferably Ar or cycloalkyl, as described above. R⁴ is particularly preferably R⁴ phenyl, o-aminophenyl, p-methoxyphenyl, 2-amino-5-bromophenyl, 2-amino-5-fluorophenyl, 2-amino-5-chlorophenyl, 2-amino-3-methylphenyl, 2-amino-5-nitrophenyl, 2-amino-4,5-dimethoxyphenyl, 3-methyl-2,5-dioxopyrrolidin-1-ylphenyl, 5-fluoro-2-(3-methyl-2,5-dioxopyrrolidin-1-yl)-phenyl, 5-bromo-2-(3-methyl-2,5-dioxopyrrolidin-1-yl)phenyl, 2-(3-methyl-2,5-dioxopyrrolidin-1-yl)-5-nitrophenyl, 4-chloro-2-(3-methyl-2,5-dioxopyrrolidin-1-yl)-phenyl, 4,5-dimethoxy-2-(3-methyl-2,5-dioxopyrrolidin-1-yl)-phenyl, 2-(1,3-dioxo-1,3-dihydroisoindol-2-yl)-phenyl, 2-(1,3-dioxo-1,3-dihydroisoindol-2-yl)-3-methylphenyl, 4-chloro-2-(1,3-dioxo-1,3-dihydroisoindol-2-yl)-phenyl, 2-(1,3-dioxo-1,3-dihydroisoindol-2-yl)-5-fluorophenyl or 4,5-dimethoxy-2-(1,3-dioxo-1,3-dihydroisoindol-2-yl)phenyl or cyclopropyl.

R⁵ is preferably hydrogen or arylalkyl. R⁵ is particularly preferably hydrogen or benzyl.

R⁵ is hydrogen or alkyl having from 1 to 10 carbon atoms, as described above. R⁶ is preferably hydrogen, methyl or ethyl.

R is A, Ar, arylalkyl, Het, C(O)—R⁴, SO₂—R⁴, C(S)N(R⁴)₂, COOR⁴ or an amino-protecting group, in which A, Ar, arylalkyl, Het and R⁴ have one of the meanings described above, and the amino-protecting group has one of the meanings given below.

In particular, R is benzyl, methyl, tert-butoxycarbonyl (Boc), 9-fluorenylmethoxycarbonyl (Fmoc) or benzoyl.

m is 1 or 2, particularly preferably 2.

o is 0, 1, 2, 3 or 4, particularly preferably 0 or 1.

The invention accordingly relates, in particular, to the compounds of the formula I in which at least one of the said radicals has one of the preferred meanings indicated above. Some preferred groups of compounds may be expressed by the following sub-formulae Ia to Ii, which conform to the formula I and in which the radicals not designated in greater detail have the meaning indicated for the formula I, but in which

| | | |
|---|---|---|
| in la | A-B | is a single bond; |
| in lb | X | is O or NH; |
| in lc | R¹ | is hydrogen, A or arylalkyl; |
| in ld | R² | is A or C(O)—R⁴, SO₂R⁵ or C(S)N(R⁵)₂; |
| in le | R² | is A, C(O)—R⁴, SO₂R⁵ or C(S)N(R⁵)₂, and |
| | R⁴ | is A, Ar or cycloalkyl; |
| in lf | X | is O, |
| | R¹ | is hydrogen or arylalkyl, |
| | R² | is A, C(O)—R⁴ or C(S)N(R⁵)₂, |
| | R⁴ | is Ar or cycloalkyl, and |
| | R⁵ | is hydrogen or arylalkyl; |
| in lg | X | is NH, |
| | R¹ | is A or arylalkyl, |
| | R² | is C(O)—R⁴ or SO₂—R⁵, |
| | R⁴ | is A or Ar, and |
| | R⁵ | is arylalkyl; |
| in lh | A-B | is a single bond, |
| | X | is O, |
| | R¹ | is hydrogen or arylalkyl, |
| | R² | is A, C(O)—R⁴ or C(S)N(R⁵)₂, |
| | R⁴ | is Ar or cycloalkyl and |
| | R⁵ | is hydrogen or arylalkyl; |
| in li | A-B | is a single bond, |
| | X | is NH, |
| | R¹ | is A or arylalkyl, |
| | R² | is C(O)—R⁴ or SO₂—R⁵, |
| | R⁴ | is A or Ar, and |
| | R⁵ | is arylalkyl. |

In particular, the invention relates to the compounds

a) 2′-benzyl-2′-azabicyclo[2.2.1]hept-7′-ylmethyl 2-amino-5-bromobenzoate,

b) 2-benzyl-7-methoxymethyl-2-azabicyclo[2.2.1]heptane,

c) 2′-benzyl-2′-azabicyclo[2.2.1]hept-7′-ylmethyl cyclopropanecarboxylate,

d) 2″-benzyl-2″-azabicyclo[2.2.1]hept-7″-ylmethyl 5-bromo-2-(3′-methyl-2′,5′-dioxopyrrolidin-1′-yl)benzoate,

e) 2″-benzyl-2″-azabicyclo[2.2.1]hept-7″-ylmethyl 2-(1,3-dioxo-1,3-dihydroisoindol-2-yl)-4,5-dimethoxybenzoate,

f) 2-azabicyclo[2.2.1]hept-7-ylmethyl benzoate,

g) N-(2-benzyl-2-azabicyclo[2.2.1]hept-7-ylmethyl)acetamide,

h) N-(2-benzyl-2-azabicyclo[2.2.1]hept-7-ylmethyl)-1-phenylmethanesulfonamide,

i) 2-methyl-2-azabicyclo[2.2.1]hept-7-ylmethyl benzoate or

j) O-(2-benzyl-2-azabicyclo[2.2.1]hept-7-ylmethyl) N-benzylthiocarbamate.

The compounds of the formula I and also the starting materials for their preparation are, in addition, prepared by methods known per se, as described in the literature (for example in the standard works, such as Houben-Weyl, Methoden der Organischen Chemie [Methods of Organic Chemistry], Georg Thieme Verlag, Stuttgart; Organic Reactions, John Wiley & Sons, Inc., New York), to be precise under reaction conditions as are known and suitable for the said reactions. Use can also be made here of variants which are known per se, but are not mentioned here in greater detail.

The starting materials for the claimed process can also be formed in situ by not isolating them from the reaction mixture, but instead immediately converting them further into the compounds of the formula I. On the other hand, it is possible to carry out the reaction in steps.

In the compounds of the formulae III and IV, the radical L is preferably Cl or Br; however, it may alternatively be I, OH or preferably a reactively functionally modified OH group, in particular alkylsulfonyloxy having 1-6 carbon atoms (for example methanesulfonyloxy) or arylsulfonyloxy having 6-10 (for example benzenesulfonyloxy, p-toluenesulfonyloxy or 1- or 2-naphthalenesulfonyloxy) or alternatively trichloromethoxy, alkoxy, such as, for example, methoxy, ethoxy, propoxy or butoxy, furthermore also phenoxy.

The compounds of the formula I in which X=O and R²=C(O)—R⁴ can preferably be obtained by reacting compounds of the formula II in which X=O with compounds of the formula III.

The starting materials of the formula II are generally known; the compounds of the formula II which are not known can easily be prepared analogously to the known compounds. (2-Benzyl-2-azabicyclo[2.2.1]hept-7-yl)methanol is disclosed in WO 92/05172.

The substituent R in the formula II can, as described above, also be an amino-protecting group. After reaction of the corresponding compound of the formula II with a compound of the formula III, the amino-protecting group can be cleaved off to give the free amine (R¹=hydrogen) and then, if desired, converted into another substituent R¹.

The term “amino protecting group” is generally known and relates to groups which are suitable for protecting (blocking) an amino group against chemical reactions. Typical of such groups are, in particular, unsubstituted or substituted acyl, aryl, aralkoxymethyl or aralkyl groups. Since the amino protecting groups are removed after the desired reaction (or synthesis sequence), their type and size is furthermore not crucial; however, preference is given to those having 1-20 carbon atoms. The term “acyl group” is to be understood in the broadest sense in connection with the present process. It includes acyl groups derived aliphatic, araliphatic, alicyclic, aromatic and heterocyclic carboxylic acids or sulfonic acids, as well as, in particular, alkoxycarbonyl, alkenyloxycarbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as phenoxyacetyl; alkoxycarbonyl, such as methoxycarbonyl, ethoxycarbonyl, 2,2,2-trichloroethoxycarbonyl, Boc and 2-iodoethoxycarbonyl; alkenyloxycarbonyl, such as allyloxycarbonyl (Aloc), aralkoxycarbonyl, such as CBZ (synonymous with Z), 4-methoxybenzyloxycarbonyl (MOZ), 4-nitrobenzyloxycarbonyl and 9-fluorenylmethoxycarbonyl (Fmoc); 2-(phenylsulfonyl)ethoxycarbonyl; trimethylsilylethoxycarbonyl (Teoc), and arylsulfonyl, such as 4-methoxy-2,3,6-trimethylphenylsulfonyl (Mtr). Preferred amino protecting groups are Boc, Fmoc and Aloc, furthermore Z and acetyl.

The term “hydroxyl-protecting group” is likewise generally known and relates to groups which are suitable for protecting a hydroxyl group against chemical reactions. Typical of such groups are the above-mentioned unsubstituted or substituted aryl, aralkyl, aroyl or acyl groups, furthermore also alkyl groups, alkyl-, aryl- and aralkylsilyl groups, and O,O- and O,S-acetals. The nature and size of the hydroxyl-protecting groups is not crucial since they are removed again after the desired chemical reaction or synthesis sequence; preference is given to groups having 1-20 carbon atoms, in particular 1-10 carbon atoms. Examples of hydroxyl-protecting groups are, inter alia, aralkyl groups, such as benzyl, 4-methoxybenzyl and 2,4-di-methoxybenzyl, aroyl groups, such as benzoyl and p-nitrobenzoyl, acyl groups, such as acetyl and pivaloyl, p-toluenesulfonyl, alkyl groups, such as methyl and tert-butyl, but also allyl, alkylsilyl groups, such as trimethylsilyl (TMS), triisopropylsilyl (TIPS), tert-butyidimethylsilyl (TBS) and triethylsilyl, trimethylsilylethyl, aralkylsilyl groups, such as tert-butyidiphenylsilyl (TBDPS), cyclic acetals, such as isopropylidene acetal, cyclopentylidene acetal, cyclohexylidene acetal, benzylidene acetal, p-methoxybenzylidene acetal and o,p-dimethoxybenzylidene acetal, acyclic acetals, such as tetrahydropyranyl (Thp), methoxymethyl (MOM), methoxyethoxymethyl (MEM), benzyloxymethyl (BOM) and methylthiomethyl (MTM). Particularly preferred hydroxyl-protecting groups are benzyl, acetyl, tert-butyl and TBS.

The liberation of the compounds of the formula I from their functional derivatives is known from the literature for the protecting group used in each case (for example T. W. Greene, P. G. M. Wuts, Protective Groups in Organic Chemistry, 2nd Edn., Wiley, New York 1991 or P. J. Kocienski, Protecting Groups, 1 st Edn., Georg Thieme Verlag, Stuttgart—New York, 1994). Use may also be made here of variants which are known per se, but are not mentioned here in greater detail.

Activated acids of the formula III are commercially available or can easily be prepared analogously to conditions which are known to the person skilled in the art by activation of the corresponding free acid of the formula III.

The reaction of the compounds of the formula II with a compound of the formula III is an esterification. Reaction conditions for an esterification are known to the person skilled in the art.

The esterification can be carried out, for example, by reacting an acid chloride of the formula III with an alcohol of the formula II in the presence of a base at temperatures between 0° and 100° C., preferably between 200 and 50° C.

Examples of suitable inert solvents are hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, N-methylpyrrolidone (NMP), dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

The compounds of the formula I in which X=O and R²=C(O)—R⁴ in which R⁴ is a phenyl group which contains a free amino group and is optionally monosubstituted or polysubstituted by D can preferably be obtained by reacting compounds of the formula II

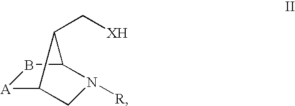

in which A-B is a single or double bond,

X is O, and

R is A, Ar, arylalkyl, Het, C(O)—R⁴, SO₂—R⁴, C(S)N(R⁴)₂, COOR⁴ or an amino-protecting group,

with a compound of the formula II-A

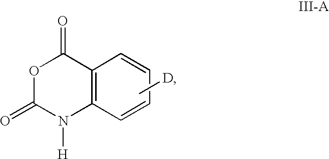

in which

D is Hal, A, OR⁶, N(R⁶)₂, NO₂, CN, COOR⁶, CON(R⁶)₂, NR⁶COR⁶, NR⁶CON(R⁶)₂, NR⁶SO₂A, COR⁶, SO₂NR⁶, S(O)ₘA, where R⁶ is hydrogen or alkyl having from 1 to 10 carbon atoms, as described above. During esterification under the conditions indicated above, free amino or hydroxyl groups must be in protected form during the reaction. After the esterification, the protecting groups can be cleaved off and the substance R converted, if desired, into a radical R¹, where R¹ can be as defined in claim 1.

The compounds of the formula I in which X=O and R²=C(O)—R⁴ in which R⁴ is a phenyl group which contains the radical 3-methyl-2,5-dioxopyrrolidin-1-yl or 1,3-dioxo-1,3-dihydroisoindol-2-yl and is optionally monosubstituted or polysubstituted by D can preferably be obtained by reacting compounds of the formula I in which X=O, R²=C(O)—R⁴, and R⁴ is a phenyl group which contains a free amino group and is optionally monosubstituted or polysubstituted by D, of the formula I-A

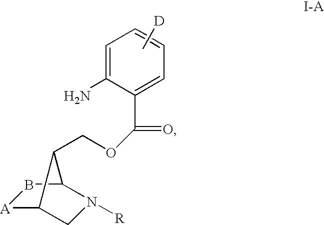

in which A-B is a single or double bond,

R is A, Ar, arylalkyl, Het, C(O)—R⁴, SO₂—R⁴, C(S)N(R⁴)₂, COOR⁴ or an amino-protecting group,

D is Hal, A, OR⁶, N(R⁶)₂, NO₂, CN, COOR⁶, CON(R⁶)₂, NR⁶COR⁶, NR⁶CON(R⁶)₂, NR⁶SO₂A, COR⁶, SO₂NR⁶ or S(O)ₘA, and

R⁶ is hydrogen or alkyl having from 1 to 10 carbon atoms,

with a compound of the formula VI or VII

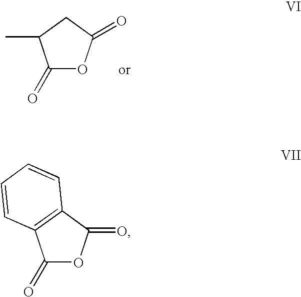

in the presence of an acid, under conditions which are known for the acylation of amines.

The reaction temperature is between about 30° and 150°, preferably between 60° and 100°, depending on the conditions used.

The reaction time is between a few minutes and several days, depending on the conditions used.

The compounds of the formula I in which X=NR³, R³ is as defined in claim 1, and R²=SO₂—R⁵ can preferably be obtained by reacting compounds of the formula II in which X=NR³ with compounds of the formula IV. The reaction is preferably carried out in the presence of a base. The reaction conditions for this sulfonation are known to the person skilled in the art, for example from P. Pinho et al., Chem. Commun. 1999, 7, 597-598.

Compounds of the formula IV are commercially available or can easily be prepared analogously to conditions which are known to the person skilled in the art.

The compounds of the formula I in which X=NR³ or O, R³ is as defined in claim 1, and R²=C(S)N(R⁵)₂ can preferably be obtained by reacting compounds of the formula II in which X=NR³ or O with compounds of the formula V. The reaction is preferably carried out in the presence of a base. The reaction conditions for this reaction are known to the person skilled in the art, for example from F. Fueloep et al, Chem. Ber. 1990, 123, 803-809.

Compounds of the formula V are commercially available or can easily be prepared analogously to conditions which are known to the person skilled in the art.

In the reactions indicated which are carried out in the presence of a base, the base used can be, for example, an alkali or alkaline earth metal hydroxide, carbonate or bicarbonate or another salt of a weak acid of the alkali or alkaline earth metals, preferably of potassium, sodium or calcium, or an organic base, such as triethylamine, dimethylaniline, pyridine, quinoline, 1,5-diazabicyclo[4.3.0]non-5-ene (DBN), 1,4-diazabicyclo[2.2.2]octane (DABCO) or 1,8-diazabicyclo[5.4.0]undec-7-ene (DBU). Suitable bases are also, in particular, those which are bound to a polymeric support, for example tris(2-aminoethyl)amine-polystyrene (NovaBiochem, Art. No. 01-64-0170).

A base of the formula I obtained can be converted into the associated acid-addition salt using an acid. Suitable acids for this reaction are those which give physiologically acceptable salts. Thus, it is possible to use inorganic acids, for example sulfuric acid, hydrohalic acids, such as hydrochloric acid or hydrobromic acid, phosphoric acids, such as orthophosphoric acid, nitric acid, sulfamic acid, furthermore organic acids, specifically aliphatic, alicyclic, araliphatic, aromatic or heterocyclic monobasic or polybasic carboxylic, sulfonic or sulfuric acids, such as formic acid, acetic acid, propionic acid, pivalic acid, diethylacetic acid, malonic acid, succinic acid, pimelic acid, fumaric acid, maleic acid, lactic acid, tartaric acid, malic acid, benzoic acid, salicylic acid, 2-phenylpropionic acid, citric acid, gluconic acid, ascorbic acid, nicotinic acid, isonicotinic acid, methane- or ethane-sulfonic acid, ethanedisulfonic acid, 2-hydroxyethanesulfonic acid, benzenesulfonic acid, p-toluenesulfonic acid, naphthalenemono- and -disulfonic acids and laurylsulfuric acid.

The free bases of the formula I can, if desired, be liberated from their salts by treatment with strong bases, such as sodium hydroxide, potassium hydroxide, sodium carbonate or potassium carbonate, so long as no further acidic groups are present in the molecule. In those cases where the compounds of the formula I have free acid groups, salt formation can likewise be achieved by treatment with bases. Suitable bases are alkali metal hydroxides, alkaline earth metal hydroxides or organic bases in the form of primary, secondary or tertiary amines.

The invention furthermore relates to the medicament active ingredients according to the invention as nicotinic acetylcholine receptor ligands for the prophylaxis or treatment of schizophrenia, depression, anxiety states, dementia, Alzheimer's disease, Lewy bodies dementia, neurodegenerative disorders, Parkinson's disease, Huntington's disease, Tourette's syndrome, learning and memory restrictions, age-induced memory impairment, amelioration of withdrawal symptoms in nicotine dependence, strokes or brain damage by toxic compounds.

The invention furthermore relates to pharmaceutical preparations comprising at least one compound of the formula I and/or one of its physiologically acceptable salts or solvates. The compounds of the formula I here can be converted into a suitable dosage form together with at least one solid, liquid and/or semi-liquid excipient or adjuvant and if desired in combination with one or more further active ingredients.

These preparations can be used as medicaments in human or veterinary medicine. Suitable excipients are organic or inorganic substances which are suitable for enteral (for example oral), parenteral or topical administration and which do not react with the novel compounds, for example water, vegetable oils, benzyl alcohols, alkylene glycols, polyethylene glycols, glycerol triacetate, gelatine, carbohydrates, such as lactose or starch, magnesium stearate, talc and Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations. The preparations indicated may be sterilised and/or comprise adjuvants, such as lubricants, preservatives, stabilisers and/or wetting agents, emulsifiers, salts for modifying the osmotic pressure, buffer substances, colorants, flavours and/or a plurality of further active ingredients, for example one or more vitamins.

The substances according to the invention are generally administered analogously to known, commercially available preparations (for example Tae-rin), preferably in doses of between about 5 mg and 100 mg, in particular between 10 and 40 mg per dosage unit. The daily dose is preferably between about 0.5 and 1 mg/kg of body weight.

The specific dose for each individual patient depends on a very wide variety of factors, for example on the efficacy of the specific compound employed, on the age, body weight, general state of health, sex, on the diet, on the time and method of administration, on the excretion rate, medicament combination and severity of the particular disorder to which the therapy applies.

Oral administration is preferred.

The above-mentioned compounds of the formula I are used for the preparation of medicaments which are employed for the treatment of disorders based on dysfunction or degeneration of nicotinic acetylcholine receptors.

The invention likewise relates to the use of compounds of the formula I according to claim 1 and/or physiologically acceptable salts or solvates thereof for the preparation of a medicament, in particular for the preparation of a medicament for the treatment of disorders in which excitation of nicotinic acetylcholine receptors results in an improvement in the clinical picture.

The invention furthermore relates to the use of compounds of the formula I according to claim 1 and/or of physiologically acceptable salts and solvates thereof for the preparation of a medicament for the prophylaxis or treatment of schizophrenia, depression, anxiety states, dementia, Alzheimer's disease, Lewy bodies dementia, neurodegenerative disorders, Parkinson's disease, Huntington's disease, Tourette's syndrome, learning and memory restrictions, age-induced memory impairment, amelioration of withdrawal symptoms in nicotine dependence, strokes or brain damage by toxic compounds.

Even without further details, it is assumed that a person skilled in the art will be able to use the above description in the broadest scope. The preferred embodiments should therefore merely be regarded as descriptive disclosure which is absolutely not limiting in any way.

Above and below, all temperatures are given in ° C. In the following examples, “conventional work-up” means that, if necessary, the solvent is removed, water is added if necessary, the pH is, if necessary, adjusted to between 2 and 10, depending on the constitution of the end product, the mixture is extracted with ethyl acetate or dichloromethane, the phases are separated, the organic phase is dried over sodium sulfate, filtered and evaporated, and the product is purified by chromatography on silica gel and/or by crystallisation. The purified compounds are, if desired, freeze-dried.

Mass spectrometry (MS): ESI (electrospray ionisation) (M+H)⁺

### EXAMPLE 1

0.2 mmol of triethylamine and 0.11 mmol of benzoyl chloride are added to a solution of 0.1 mmol of (2-benzyl-2-azabicyclo[2.2.1]hept-7-yl)methanol in 1 ml of tetrahydrofuran (THF), and the mixture is stirred at room temperature for 18 hours. The reaction mixture is subjected to conventional work-up, giving

2′-benzyl-2′-azabicyclo[2.2.1]hept-7′-ylmethyl benzoate; ESI 322.

Reaction of the free base with 0.5M HCl solution in isopropanol gives

2′-benzyl-2′-azabicyclo[2.2.1]hept-7′-ylmethyl benzoate hydrochloride.

### EXAMPLE 2

Analogously to Example 1, reaction of (2-benzyl-2-azabicyclo[2.2.1]hept-7-yl)methanol with

4-methoxybenzoyl chloride gives

2′-benzyl-2′-azabicyclo[2.2.1]hept-7′-ylmethyl 4-methoxybenzoate; ESI 352;

salt precipitation using 0.5M HCl solution gives

2′-benzyl-2′-azabicyclo[2.2.1]hept-7′-ylmethyl 4-methoxybenzoate hydrochloride,

cyclopropanecarbonyl chloride gives

2′-benzyl-2′-azabicyclo[2.2.1]hept-7′-ylmethyl cyclopropanecarboxylate; ESI 286;

salt precipitation using 0.5M HCl solution gives

2′-benzyl-2′-azabicyclo[2.2.1]hept-7′-ylmethyl cyclopropanecarboxylate hydrochloride.

### EXAMPLE 3

0.2 mmol of triethylamine and 48.4 mg of 6-bromo-1H-benzo[d]-1,3-oxazine-2,4-dione are added to a solution of 43.3 mg of (2-benzyl-2-azabicyclo[2.2.1]hept-7-ylmethanol in 1 ml of dimethylformamide (DMF), and the mixture is stirred at 80° C. for 19 hours. The reaction mixture is subjected to conventional work-up, giving

2′-benzyl-2′-azabicyclo[2.2.1]hept-7′-ylmethyl 2-amino-5-bromobenzoate; ESI 416;

salt precipitation using 0.5M HCl solution gives

2′-benzyl-2′-azabicyclo[2.2.1]hept-7′-ylmethyl 2-amino-5-bromobenzoate hydrochloride.

### EXAMPLE 4

Analogously to Example 3, reaction of (2-benzyl-2-azabicyclo[2.2.1]hept-7-yl)methanol with

6,7-dimethoxy-1H-benzo[d]-1,3-oxazine-2,4-dione gives

2′-benzyl-2′-azabicyclo[2.2.1]hept-7′-ylmethyl 2-amino-4,5-dimethoxybenzoate; ESI 397;

salt precipitation using 0.5M HCl solution gives

2′-benzyl-2′-azabicyclo[2.2.1]hept-7′-ylmethyl 2-amino-4,5-dimethoxybenzoate hydrochloride;

6-fluoro-1H-benzo[d]-1,3-oxazine-2,4-dione gives

2′-benzyl-2′-azabicyclo[2.2.1]hept-7′-ylmethyl 2-amino-5-fluorobenzoate; ESI 355;

salt precipitation using 0.5M HCl solution gives

2′-benzyl-2′-azabicyclo[2.2.1]hept-7′-ylmethyl 2-amino-5-fluorobenzoate hydrochloride;

7-chloro-1H-benzo[d]-1,3-oxazine-2,4-dione gives

2′-benzyl-2′-azabicyclo[2.2.1]hept-7′-ylmethyl 2-amino-4-chlorobenzoate; ESI 372;

salt precipitation using 0.5M HCl solution gives

2′-benzyl-2′-azabicyclo[2.2.1]hept-7′-ylmethyl 2-amino-4-chlorobenzoate hydrochloride;

8-methyl-1H-benzo[d]-1,3-oxazine-2,4-dione gives

2′-benzyl-2′-azabicyclo[2.2.1]hept-7′-ylmethyl 2-amino-3-methylbenzoate; ESI 351;

salt precipitation using 0.5M HCl solution gives

2′-benzyl-2′-azabicyclo[2.2.1]hept-7′-ylmethyl 2-amino-3-methylbenzoate hydrochloride;

6-nitro-1H-benzo[d]-1,3-oxazine-2,4-dione gives

2′-benzyl-2′-azabicyclo[2.2.1]hept-7′-ylmethyl 2-amino-5-nitrobenzoate; ESI 382;

salt precipitation using 0.5M HCl solution gives

2′-benzyl-2′-azabicyclo[2.2.1]hept-7′-ylmethyl 2-amino-5-nitrobenzoate hydrochloride;

1H-benzo[d]-1,3-oxazine-2,4-dione gives

2′-benzyl-2′-azabicyclo[2.2.1]hept-7′-ylmethyl 2-aminobenzoate; ESI 337;

salt precipitation using 0.5M HCl solution gives

2′-benzyl-2′-azabicyclo[2.2.1]hept-7′-ylmethyl 2-aminobenzoate hydrochloride.

### EXAMPLE 5

0.190 mmol of 3-methyldihydrofuran-2,5-dione is added to a solution of 0.173 mmol of 2′-benzyl-2′-azabicyclo[2.2.1]hept-7′-ylmethyl 2-amino-5-bromobenzoate, obtained in accordance with Example 3, in 1 ml of acetic acid, and the mixture is heated at 80° C. for 12 hours. The mixture is subjected to conventional work-up, giving

2″-benzyl-2″-azabicyclo[2.2.1]hept-7″-ylmethyl 5-bromo-2-(3′-methyl-2′,5′-dioxopyrrolidin-1′-yl)benzoate; ESI 512;

salt precipitation using 0.5M HCl solution gives

2″-benzyl-2″-azabicyclo[2.2.1]hept-7″-ylmethyl 5-bromo-2-(3′-methyl-2′,5′-dioxopyrrolidin-1′-yl)benzoate hydrochloride.

### EXAMPLE 6

Analogously to Example 5, reaction of 3-methyldihydrofuran-2,5-dione with

2′-benzyl-2′-azabicyclo[2.2.1]hept-7′-ylmethyl 2-amino-4,5-dimethoxybenzoate gives

2″-benzyl-2″-azabicyclo[2.2.1]hept-7″-ylmethyl 4,5-dimethoxy-2-(3′-methyl-2′,5′-dioxopyrrolidin-1′-yl)benzoate; ESI 494;

salt precipitation using 0.5M HCl solution gives

2″-benzyl-2″-azabicyclo[2.2.1]hept-7″-ylmethyl 4,5-dimethoxy-2-(3′-methyl-2′,5′-dioxopyrrolidin-1′-yl)benzoate hydrochloride;

2′-benzyl-2′-azabicyclo[2.2.1]hept-7′-ylmethyl 2-amino-5-fluorobenzoate gives

2″-benzyl-2″-azabicyclo[2.2.1]hept-7″-ylmethyl 5-fluoro-2-(3′-methyl-2′,5′-dioxopyrrolidin-1′-yl)benzoate; ESI 452;

salt precipitation using 0.5M HCl solution gives

2″-benzyl-2″-azabicyclo[2.2.1]hept-7″-ylmethyl 5-fluoro-2-(3′-methyl-2′,5′-dioxopyrrolidin-1′-yl)benzoate hydrochloride;

2′-benzyl-2′-azabicyclo[2.2.1]hept-7′-ylmethyl 2-amino-4-chlorobenzoate gives

2″-benzyl-2″-azabicyclo[2.2.1]hept-7″-ylmethyl 4-chloro-2-(3′-methyl-2′,5′-dioxopyrrolidin-1′-yl)benzoate; ESI 468;

salt precipitation using 0.5M HCl solution gives

2″-benzyl-2″-azabicyclo[2.2.1]hept-7″-ylmethyl 4-chloro-2-(3′-methyl-2′,5′-dioxopyrrolidin-1′-yl)benzoate hydrochloride;

2′-benzyl-2′-azabicyclo[2.2.1]hept-7′-ylmethyl 2-amino-3-methylbenzoate gives

2″-benzyl-2″-azabicyclo[2.2.1]hept-7″-ylmethyl 3-methyl-2-(3′-methyl-2′,5′-dioxopyrrolidin-1′-yl)benzoate; ESI 448;

salt precipitation using 0.5M HCl solution gives

2″-benzyl-2″-azabicyclo[2.2.1]hept-7″-ylmethyl 3-methyl-2-(3′-methyl-2′,5′-dioxopyrrolidin-1′-yl)benzoate hydrochloride;

2′-benzyl-2′-azabicyclo[2.2.1]hept-7′-ylmethyl 2-amino-5-nitrobenzoate gives

2″-benzyl-2″-azabicyclo[2.2.1]hept-7″-ylmethyl 2-(3′-methyl-2′,5′-dioxopyrrolidin-1′-yl)-5-nitrobenzoate; ESI 479;

salt precipitation using 0.5M HCl solution gives

2″-benzyl-2″-azabicyclo[2.2.1]hept-7″-ylmethyl 2-(3′-methyl-2′,5′-dioxopyrrolidin-1′-yl)-5-nitrobenzoate hydrochloride;

2′-benzyl-2′-azabicyclo[2.2.1]hept-7′-ylmethyl 2-aminobenzoate gives

2″-benzyl-2″-azabicyclo[2.2.1]hept-7″-ylmethyl 2-(3′-methyl-2′,5′-dioxopyrrolidin-1′-yl)benzoate; ESI 434;

salt precipitation using 0.5M HCl solution gives

2″-benzyl-2″-azabicyclo[2.2.1]hept-7″-ylmethyl 2-(3′-methyl-2′,5′-dioxopyrrolidin-1′-yl)benzoate hydrochloride.

### EXAMPLE 7

Analogously to Example 5, reaction of isobenzofuran-1,3-dione with

2′-benzyl-2′-azabicyclo[2.2.1]hept-7′-ylmethyl 2-amino-4,5-dimethoxybenzoate gives

2″-benzyl-2″-azabicyclo[2.2.1]hept-7″-ylmethyl 2-(1,3-dioxo-1,3-dihydroisoindol-2-yl)-4,5-dimethoxybenzoate; ESI 528;

salt precipitation using 0.5M HCl solution gives

2″-benzyl-2″-azabicyclo[2.2.1]hept-7″-ylmethyl 2-(1,3-dioxo-1,3-dihydroisoindol-2-yl)-4,5-dimethoxybenzoate hydrochloride;

2′-benzyl-2′-azabicyclo[2.2.1]hept-7′-ylmethyl 2-amino-5-fluorobenzoate gives

2″-benzyl-2″-azabicyclo[2.2.1]hept-7″-ylmethyl 2-(1,3-dioxo-1,3-dihydroisoindol-2-yl)-5-fluorobenzoate; ESI 486;

salt precipitation using 0.5M HCl solution gives

2″-benzyl-2″-azabicyclo[2.2.1]hept-7″-ylmethyl 2-(1,3-dioxo-1,3-dihydroisoindol-2-yl)-5-fluorobenzoate hydrochloride;

2′-benzyl-2′-azabicyclo[2.2.1]hept-7′-ylmethyl 2-amino-4-chlorobenzoate gives

2″-benzyl-2″-azabicyclo[2.2.1]hept-7″-ylmethyl 4-chloro-2-(1,3-dioxo-1,3-dihydroisoindol-2-yl)benzoate; ESI 502;

salt precipitation using 0.5M HCl solution gives

2″-benzyl-2″-azabicyclo[2.2.1]hept-7″-ylmethyl 4-chloro-2-(1,3-dioxo-1,3-dihydroisoindol-2-yl)benzoate hydrochloride;

2′-benzyl-2′-azabicyclo[2.2.1]hept-7′-ylmethyl 2-amino-3-methylbenzoate gives

2″-benzyl-2″-azabicyclo[2.2.1]hept-7″-ylmethyl 2-(1,3-dioxo-1,3-dihydroisoindol-2-yl)-3-methylbenzoate; ESI 482;

salt precipitation using 0.5M HCl solution gives

2″-benzyl-2″-azabicyclo[2.2.1]hept-7″-ylmethyl 2-(1,3-dioxo-1,3-dihydroisoindol-2-yl)-3-methylbenzoate hydrochloride;

2′-benzyl-2′-azabicyclo[2.2.1]hept-7′-ylmethyl 2-aminobenzoate gives

2″-benzyl-2″-azabicyclo[2.2.1]hept-7″-ylmethyl 2-(1,3-dioxo-1,3-dihydroisoindol-2-yl)benzoate; ESI 468;

salt precipitation using 0.5M HCl solution gives

2″-benzyl-2″-azabicyclo[2.2.1]hept-7″-ylmethyl 2-(1,3-dioxo-1,3-dihydroisoindol-2-yl)benzoate hydrochloride.

### EXAMPLE 8

Analogously to Example 3, reaction of (2-benzyl-2-azabicyclo[2.2.1]hept-7-yl)methylamine with

6-bromo-1H-benzo[d]-1,3-oxazine-2,4-dione gives

2-amino-N-(2′-benzyl-2′-azabicyclo[2.2.1]hept-7′-ylmethyl)-5-bromobenzamide; ESI 415;

salt precipitation using 0.5M HCl solution gives

2-amino-N-(2′-benzyl-2′-azabicyclo[2.2.1]hept-7′-ylmethyl)-5-bromobenzamide hydrochloride.

### EXAMPLE 9

Analogously to Example 1, reaction of (2-benzyl-2-azabicyclo[2.2.1]hept-7-yl)methylamine with

acetyl chloride gives

N-(2-benzyl-2-azabicyclo[2.2.1]hept-7-ylmethyl)acetamide; ESI 259;

salt precipitation using 0.5M HCl solution gives

N-(2-benzyl-2-azabicyclo[2.2.1]hept-7-ylmethyl)acetamide hydrochloride.

### EXAMPLE 10

5 mg of Pd/C, 10%, are added to a solution of 0.18 mmol of 2′-benzyl-2′-azabicyclo[2.2.1]hept-7′-ylmethyl benzoate, obtained from Example 1, in 2 ml of methanol, and the mixture is stirred for 12 hours at room temperature under a hydrogen atmosphere. The solution is filtered, and the solvent is removed, giving 2-azabicyclo[2.2.1]hept-7-ylmethyl benzoate; ESI 232. Salt precipitation using 0.5M HCl solution gives 2-azabicyclo[2.2.1]hept-7-ylmethyl benzoate hydrochloride.

0.105 mmol of formaldehyde (37% in water) and 0.210 mmol of formic acid are added to a solution of 0.07 mmol of 2-azabicyclo[2.2.1]hept-7-ylmethyl benzoate in 1 ml of chloroform, and the mixture is stirred at 60° C. for 12 hours. The mixture is subjected to conventional work-up, giving 2-methyl-2-azabicyclo[2.2.1]hept-7-ylmethyl benzoate; ESI 246. Salt precipitation using 0.5M HCl solution gives 2-methyl-2-azabicyclo[2.2.1]hept-7-ylmethyl benzoate hydrochloride.

### EXAMPLE 11

Reaction of 2-benzyl-2-azabicyclo[2.2.1]hept-7-yl)methanol with sodium hydride and methyl iodide gives 2-benzyl-7-methoxymethyl-2-azabicyclo-[2.2.1]heptane; ESI 232. Salt precipitation using 0.5M HCl solution gives 2-benzyl-7-methoxymethyl-2-azabicyclo[2.2.1]heptane hydrochloride.

### EXAMPLE 12

54.3 mg of DIEA resin (DIEA=N,N-diisopropylaminomethyl-polystyrene) (Argonaut Tech, Art. No. 800279) and 0.06 mmol of phenylmethanesulfonyl chloride are added to a solution of 0.05 mmol of (2-benzyl-2-azabicyclo-[2.2.1]hept-7-yl)methylamine in 1 ml of absolute DMF, and the mixture is stirred at room temperature for 3 hours. 81 mg of tris(2-aminoethyl)amine-polystyrene (NovaBiochem, Art. No. 01-64-0170) are then added, and the mixture is stirred at room temperature for a further 3 hours. The solution is filtered, and the solvent is removed, giving N-(2-benzyl-2-azabicyclo[2.2.1]-hept-7-ylmethyl)-1-phenylmethanesulfonamide; ESI 372. Salt precipitation using 0.5M HCl solution gives N-(2-benzyl-2-azabicyclo[2.2.1]hept-7-ylmethyl)-1-phenylmethanesulfonamide hydrochloride.

### EXAMPLE 13

0.110 mmol of isothiocyanatomethylbenzene is added at room temperature to a solution of 0.1 mmol of (2-benzyl-2-azabicyclo[2.2.1]hept-7-yl)methanol in 2 ml of tetrahydrofuran (THF). The mixture is stirred at room temperature for 6 hours, and 0.30 mmol of tris(2-aminoethyl)amine-polystyrene (NovaBiochem, Art. No. 01-64-0170) and 0.30 mmol of methyl isocyanate-polystyrene (NovaBiochem, Art. No. 01-64-0169) are added, and the mixture is stirred at 50° C. for 12 hours. The solution is filtered, and the solvent is removed, giving O-(2-benzyl-2-azabicyclo[2.2.1]hept-7-ylmethyl) N-benzylthiocarbamate; ESI 368. Salt precipitation using 0.5M HCl solution gives O-(2-benzyl-2-azabicyclo[2.2.1]hept-7-ylmethyl) N-benzylthiocarbamate hydrochloride.

The following examples relate to pharmaceutical preparations

### EXAMPLE A

### Injection Vials

A solution of 100 g of an active ingredient of the formula I and 5 g of disodium hydrogenphosphate in 31 of bidistilled water is adjusted to pH 6.5 using 2N hydrochloric acid, sterile filtered, transferred into injection vials, lyophilised and sealed under sterile conditions. Each injection vial contains 5 mg of active ingredient.

### EXAMPLE B

### Suppositories

A mixture of 20 g of an active ingredient of the formula I is melted with 100 g of soya lecithin and 1400 g of cocoa butter, poured into moulds and allowed to cool. Each suppository contains 20 mg of active ingredient.

### EXAMPLE C

### Solution

A solution is prepared from 1 g of an active ingredient of the formula I, 9.38 g of NaH₂PO₄×2H₂O, 28.48 g of Na₂HPO₄×12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula I are mixed with 99.5 g of Vaseline under aseptic conditions.

### EXAMPLE E

### Tablets

A mixture of 1 kg of active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula I are introduced into hard gelatine capsules in a conventional manner in such a way that each capsule contains 20 mg of the active ingredient.

### EXAMPLE H

### Ampoules

A solution of 1 kg of active ingredient of the formula I in 601 of bidistilled water is sterile filtered, transferred into ampoules, lyophilised under sterile conditions and sealed under sterile conditions. Each ampoule contains 10 mg of active ingredient.

1. Compounds of the general formula I 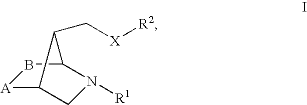
in which
A-B is a single or double bond,
X is O, NR³ or S,
R¹ is hydrogen, A, Ar, arylalkyl, Het, C(O)—R⁴, SO₂—R⁴, C(S)N(R⁴)₂ or COOR⁴,
R² is A, Ar, arylalkyl, Het, C(O)—R⁴, SO₂—R⁵, C(S)N(R⁵)₂ or COOR⁴,
R³ to R⁵ are each, independently of one another, hydrogen, A, cycloalkyl, Ar or arylalkyl,
R⁶ is hydrogen or A,
A is a linear or branched alkyl group having from 1 to 10 carbon atoms,
Ar is phenyl, naphthyl or biphenyl, each of which is unsubstituted or monosubstituted or polysubstituted by Hal, A, OR⁶, N(R⁶)₂, NO₂, CN, COOR⁶, CON(R⁶)₂, NR⁶COR⁶, NR⁶CON(R⁶)₂, NR⁶SO₂A, COR⁶, SO₂NR⁶, S(O)ₘA or Het¹,
arylalkyl is arylalkyl having 7-14 carbon atoms,
cycloalkyl is cycloalkyl having from 3 to 10 carbon atoms,
Hal is F, Cl, Br or I,
Het is a saturated, unsaturated or aromatic monocyclic or bicyclic heterocyclic radical having from 5 to 10 ring members which may contain from 1 to 4 N and/or from 1 to 4 S and/or from 1 to 4 O atoms, and where the heterocyclic radical may be monosubstituted, disubstituted or trisubstituted by Hal, A, —[C(R⁶)₂]ₒ—Ar, —[C(R⁶)₂]ₒ-cycloalkyl, OR⁶, N(R⁶)₂, NO₂, CN, COOR⁶, CON(R⁶)₂, NR⁶COA, NR⁶CON(R⁶)₂, NR⁶SO₂A, COR⁶, SO₂NR⁶ or S(O)ₘA and/or carbonyl oxygen,
Het¹ is 3-methyl-2,5-dioxopyrrolidin-1-yl or 1,3-dioxo-1,3-dihydroisoindol-2-yl,
m is 1 or 2,
o is 0, 1, 2, 3 or 4,
and physiologically acceptable salts and solvates thereof.

2. Compounds of the formula I according to claim 1, in which A-B is a single bond.

3. Compounds of the formula I according to claim 1 or 2, in which X is O or NH.

4. Compounds of the formula I according to one or more of claims 1 to 3, in which R¹ is hydrogen, A or arylalkyl.

5. Compounds of the formula I according to one or more of claims 1 to 4, in which R² is A, C(O)—R⁴, SO₂R⁵ or C(S)N(R⁵)₂.

6. Compounds of the formula I according to one or more of claims 1 to 5, in which R⁴ is A, Ar or cycloalkyl.

7. Compounds of the formula I according to one or more of claims 1 to 6, in which R⁵ is arylalkyl.

8. Compounds of the formula I according to claim 1
a) 2′-benzyl-2′-azabicyclo[2.2.1]hept-7′-ylmethyl 2-amino-5-bromobenzoate,
b) 2-benzyl-7-methoxymethyl-2-azabicyclo[2.2.1]heptane,
c) 2′-benzyl-2′-azabicyclo[2.2.1]hept-7′-ylmethyl cyclopropanecarboxylate,
d) 2″-benzyl-2″-azabicyclo[2.2.1]hept-7″-ylmethyl 5-bromo-2-(3′-methyl-2′,5′-dioxopyrrolidin-1′-yl)benzoate,
e) 2″-benzyl-2″-azabicyclo[2.2.1]hept-7″-ylmethyl 2-(1,3-dioxo-1,3-dihydroisoindol-2-yl)-4,5-dimethoxybenzoate,
f) 2-azabicyclo[2.2.1]hept-7-ylmethyl benzoate,
g) N-(2-benzyl-2-azabicyclo[2.2.1]hept-7-ylmethyl)acetamide,
h) N-(2-benzyl-2-azabicyclo[2.2.1]hept-7-ylmethyl)-1-phenylmethanesulfonamide,
i) 2-methyl-2-azabicyclo[2.2.1]hept-7-ylmethyl benzoate or
j) O-(2-benzyl-2-azabicyclo[2.2.1]hept-7-ylmethyl) N-benzylthiocarbamate,
and physiologically acceptable salts and solvates thereof.

9. Process for the preparation of compounds of the formula I according to one or more of claims 1 to 8, characterised in that
a) a compound of the formula II 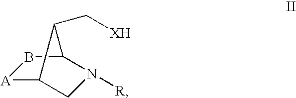
in which A-B is a single or double bond,
X is O, and
R is A, Ar, arylalkyl, Het, C(O)—R⁴, SO₂—R⁴, C(S)N(R⁴)₂, COOR⁴ or an amino-protecting group,
is reacted with a compound of the formula III
L-C(O)—R⁴ III,
in which
R⁴ is as defined in claim 1 or 6, with free amino or hydroxyl groups being in protected form during the reaction, and the protecting groups being cleaved off after esterification, and
L is Cl, Br, I or a free or reactively functionally modified OH group, and the radical R is, if desired, converted into a radical R¹, where R¹ is as defined in claim 1 or 4, or
b) a compound of the formula II 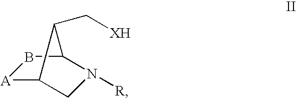
in which A-B is a single or double bond,
X is NR³,
R³ is as defined in claim 1, and
R is A, Ar, arylalkyl, Het, C(O)—R⁴, SO₂—R⁴, C(S)N(R⁴)₂, COOR⁴ or an amino-protecting group,
is reacted with a compound of the formula IV
L-SO₂—R⁵ IV,
in which R⁵ is as defined in claim 1, and
L is Cl, Br, I or a free or reactively functionally modified OH group, and the radical R is, if desired, converted into a radical R¹, where R¹ is as defined in claim 1 or 7, or
c) a compound of the formula II 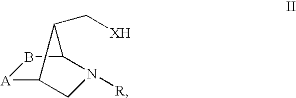
in which A-B is a single or double bond,
X is O or NR³,
R³ is as defined in claim 1, and
R is A, Ar, arylalkyl, Het, C(O)—R⁴, SO₂—R⁴, C(S)N(R⁴)₂, COOR⁴ or an amino-protecting group,
is reacted with a compound of the formula V 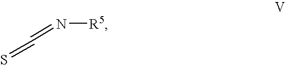
in which R⁵ is as defined in claim 1 or 7,
and the radical R is, if desired, converted into a radical R¹, where R¹ is as defined in claim 1 or 4, or
d) if desired, one of the radicals R, R¹ and/or a substituent of the aryl group is converted into another radical R, R¹ and/or a substituent of the aryl group by, for example, cleaving an OA group to form an OH group and/or converting a CHO group into a CN group and/or hydrogenating a benzyl group, and/or
a base of the formula I obtained is converted into one of its salts by treatment with an acid.

10. Compounds of the formula I according to one or more of claims 1 to 8 and physiologically acceptable salts and solvates thereof as medicament active ingredients.

11. Compounds of the formula I according to one or more of claims 1 to 8 and physiologically acceptable salts and solvates thereof as ligands of the nicotinic acetylcholine receptor.

12. Pharmaceutical preparation, characterised by a content of at least one compound of the formula I according to one or more of claims 1 to 8 and/or one of its physiologically acceptable salts or solvates.

13. Use of compounds of the formula I according to one or more of claims 1 to 8 and/or physiologically acceptable salts or solvates thereof for the preparation of a medicament.

14. Use of compounds of the formula I according to claim 1 and/or physiologically acceptable salts or solvates thereof for the preparation of a medicament, in particular for the preparation of a medicament for the treatment of disorders in which excitation of nicotinic acetylcholine receptors results in an improvement in the clinical picture.

15. Use of compounds of the formula I according to claim 1 and/or of physiologically acceptable salts and solvates thereof for the preparation of a medicament for the prophylaxis or treatment of schizophrenia, depression, anxiety states, dementia, Alzheimer's disease, Lewy bodies dementia, neurodegenerative disorders, Parkinson's disease, Huntington's disease, Tourette's syndrome, learning and memory restrictions, age-induced memory impairment, amelioration of withdrawal symptoms in nicotine dependence, strokes or brain damage by toxic compounds.
